(19) United States
(12) Patent Application Publication
(10) Pub. No.: US 2024/0399021 A1
(43) Pub. Date: Dec. 5, 2024

(54) ANTIMICROBIAL COMPOSITION AND METHOD FOR MAKING THE SAME

(71) Applicant: WACKER CHEMIE AG, Munich (DE)

(72) Inventors: Ufuk KARABIYIK, Ann Arbor, MI (US); Aaron BUSH, Jackson, MI (US); Lindsey MAXWELL, Murfreesboro, TN (US); Nicholas WHITE, Whitmore Lake, MI (US)

(73) Assignee: WACKER CHEMIE AG, Munich (DE)

(21) Appl. No.: 18/700,760

(22) PCT Filed: Oct. 13, 2021
(86) PCT No.: PCT/EP2021/078368

§ 371 Date: Apr. 12, 2024

(51) Int. Cl. A61L 26/00; A01N 25/10; A01N 59/16; A01P 1/00

(52) U.S. Cl. CPC A61L 26/0019; A01N 25/10; A01N 59/16; A01P 1/00; A61L 26/0004; A61L 26/0066; A61L 2300/104; A61L 2300/404

(57) ABSTRACT

An antimicrobial composition includes a component (A). Component (A) includes an organopolysiloxane having one or more groups comprising a silicon atom bonded to a hydrogen atom. The antimicrobial composition includes a silver-containing antimicrobial agent and an excipient. The anti-microbial composition has a discontinuous phase and the antimicrobial agent and the excipient are present in the discontinuous phase.

### BACKGROUND

The invention relates to an antimicrobial composition, methods for making such compositions, and uses for the same.

Wound care dressings often contain an adhesive layer that includes a silver-containing compound and other anti-microbial agents to help prevent infections and promote wound healing. However, over time, such silver-containing compounds disassociate. The resulting disassociation creates silver ions, which can be reduced and oxidized leading to discoloration of the dressing. Such discoloration is not desirable, particularly in a hospital setting.

Therefore, it would be desirable to provide a composition that can overcome the aforementioned deficiencies. A method for making such a composition would also be beneficial.

### BRIEF SUMMARY

Embodiments of an antimicrobial composition are provided.

In an embodiment, the antimicrobial composition comprises a component (A). Component (A) includes an organopolysiloxane having one or more groups comprising a silicon atom bonded to a hydrogen atom. The antimicrobial composition also includes a silver-containing antimicrobial agent and an excipient. The antimicrobial composition has a discontinuous phase and the antimicrobial agent and the excipient are present in the discontinuous phase.

Preferably, the silver-containing antimicrobial agent is silver salt.

In some embodiments, the excipient has an average molecular weight of 300 g/mol or more. In an embodiment, the average molecular weight of the excipient is 300-2,000 g/mol. In certain embodiments, the excipient is an alcohol, a glycol, a polyether, or an organopolysiloxane polyoxyalkylene. In one such embodiment, the excipient is a polyether having an average molecular weight of 300-2,000 g/mol. In another embodiment, the average molecular weight of the excipient is 300-1000 g/mol.

In certain embodiments, the excipient inhibits the silver-containing antimicrobial agent from forming elemental silver or silver oxide in the presence of air having a relative humidity of 50% at 20° C.

In some embodiments, the weight percent of the excipient is greater than the weight percent of the silver-containing antimicrobial agent, based on the total weight of the composition. In an embodiment, the weight percent of the excipient in the composition is 2 or more times greater than that of the silver-containing antimicrobial agent. Preferably, the weight percent of the excipient is 2 to 10 times greater than that of the silver-containing antimicrobial agent.

In other embodiments, the antimicrobial composition comprises 0.5 wt % or more of the excipient, based on the total weight of the composition. In one such embodiment, the antimicrobial composition comprises from 0.5 to 15 wt % of the excipient, based on the total weight of the composition.

In some embodiments, the antimicrobial composition further comprises a hydrophilic additive compound.

In other embodiments, the antimicrobial composition does not exhibit an observable change in color after exposure to air having a relative humidity of 50% at 20° C. for 24 hours.

In other embodiments, the antimicrobial composition further comprises a component (B) that includes an organopolysiloxane compound having one or more terminal groups comprising a carbon-carbon multiple bond.

In some embodiments, the antimicrobial composition has a continuous phase comprising an polysiloxane network formed by the product of combining component (A) and component (B). In some of these embodiments, the average molecular weight of the excipient is 300-1000 g/mol.

In other embodiments, the antimicrobial composition further comprises a hydrosilyation catalyst provided as a portion of component (A) or component (B).

In some embodiments, the antimicrobial composition further comprises a component (C). In an embodiment, component (C) comprises an organopolysiloxane and a filler.

In some embodiments, a gel adhesive may comprise the antimicrobial composition.

### DETAILED DESCRIPTION

It is to be understood that the invention may assume various alternative orientations and step sequences, except where expressly specified to the contrary. It is also to be understood that the specific materials, compositions, articles, and methods described in the following specification are simply exemplary embodiments of the inventive concepts. Hence, specific properties, conditions, or other physical characteristics relating to the embodiments disclosed are not to be considered as limiting, unless expressly stated otherwise.

In certain embodiments, an antimicrobial composition is provided. The antimicrobial composition is suitable for use in wound care dressings. For example, the composition may be utilized to provide an adhesive portion for the wound care dressing. However, the antimicrobial composition is not limited to wound care applications and can be utilized in other applications where inhibiting the growth of microbes is desired. Such applications may be of the medical or non-medical variety.

The antimicrobial composition comprises a component (A). Component (A) includes an organopolysiloxane having one or more groups comprising a silicon atom bonded to a hydrogen atom. A silicon atom bonded to a hydrogen atom may also be referred to herein as Si-bonded hydrogen or by using the designation “SiH.” In some embodiments, at least one of the one or more groups comprising a silicon atom bonded to a hydrogen atom is a terminal group. In other embodiments, at least one of the one or more groups comprising a silicon atom bonded to a hydrogen atom is a pendant group. In still other embodiments, the organopolysiloxane of component (A) has two or more groups comprising a silicon atom bonded to a hydrogen atom and at least one group of the two or more groups is a terminal group and at least one group of the two or more groups is a pendant group. In some embodiments, component (A) may comprise a mixture of the organopolysiloxanes described above. For example, in an embodiment, component (A) may comprise a mixture of organopolysiloxanes and the mixture may comprise an organopolysiloxane having at least one terminal group comprising a silicon atom bonded to a hydrogen atom and an organopolysiloxane having at least one pendant group comprising a silicon atom bonded to a hydrogen atom. Additional organopolysiloxanes may also be suitable for use in component (A).

Preferably, the organopolysiloxane of component (A) has two or more Si-bonded hydrogen atoms, is linear, cyclic, or branched, and composed of units of the general formula (I)

R⁴_{c}H_{d}SiO_{(4-c-d)/2} (I)

where
R⁴ independently at each occurrence, is a radical free from aliphatic carbon-carbon multiple bonds,
c is 0, 1, 2, or 3, and
d is 0, 1, or 2,
   with the proviso that the sum of c+d is less than or equal to 3 and there are at least two Si-bonded hydrogen atoms per molecule.

In some embodiments, R⁴ may comprise one or more monovalent or polyvalent radicals, in which case the polyvalent radicals, such as divalent, trivalent, and tetravalent radicals, for example, join two or more, such as two, three, or four, for instance, siloxy units of the formula (I) to one another.

In other embodiments, R⁴ may be a monovalent radical of the group comprising —F, —Cl, —Br, OR⁶, —CN, —SCN, —NCO, and SiC-bonded, substituted or unsubstituted hydrocarbon radicals which may be interrupted by oxygen atoms or by the group —C(O)—, and also divalent radicals Si-bonded on both sides in accordance with formula (I). If R⁴ comprises SiC-bonded, substituted hydrocarbon radicals, preferred substituents include halogen atoms, phosphorus-containing radicals, cyano radicals, —OR⁶, —NR⁶—, —NR⁶₂, —NR⁶—C(O)—NR⁶₂, —C(O)—NR⁶₂,—C(O) R⁶, —C(O)OR⁶, —SO₂-Ph, and —C₆F₅. In such embodiments, R⁶, independently at each occurrence, identically or differently, denotes a hydrogen atom or a monovalent hydrocarbon radical having 1 to 20 carbon atoms, and Ph is the phenyl radical.

Further embodiments of R⁴ include alkyl radicals, such as the methyl, ethyl, n-propyl, isopropyl, n-butyl, isobutyl, tert-butyl, n-pentyl, isopentyl, neopentyl, tert-pentyl radical, hexyl radicals, such as the n-hexyl radical, heptyl radicals, such as the n-heptyl radical, octyl radicals, such as the n-octyl radical and isooctyl radicals, such as the 2,2,4-trimethylpentyl radical, nonyl radicals, such as the n-nonyl radical, decyl radicals, such as the n-decyl radical, dodecyl radicals, such as the n-dodecyl radical, and octadecyl radicals, such as the n-octadecyl radical, cycloalkyl radicals, such as cyclopentyl, cyclohexyl, cycloheptyl, and methylcyclohexyl radicals, aryl radicals, such as the phenyl, naphthyl, anthryl, and phenanthryl radical, alkaryl radicals, such as o-, m-, p-tolyl radicals, xylyl radicals, and ethylphenyl radicals, and aralkyl radicals, such as the benzyl radical, the α- and the ß-phenylethyl radical.

When R⁴ is a substituted radical, suitable examples include are haloalkyl radicals, such as the 3,3,3-trifluoro-n-propyl radical, the 2,2,2,2′,2′,2′-hexafluoroisopropyl radical, the heptafluoroisopropyl radical, haloaryl radicals, such as the o-, m-, and p-chlorophenyl radical, —(CH₂)—N(R⁶)C(O)NR⁶₂, —(CH₂)ₒ—C(O)NR⁶₂, —(CH₂)ₒ—C(O)R⁶, —(CH₂)ₒ—C(O)OR⁶, —(CH₂)ₒ—C(O)NR⁶₂, —(CH₂)—C(O)—(CH₂)ₚC(O)CH₃, —(CH₂)—O—CO—R⁶, —(CH₂)—NR⁶—(CH₂)ₚ—NR⁶₂, —(CH₂)ₒ—O—(CH₂)ₚCH(OH)CH₂OH, —(CH₂)ₒ(OCH₂CH₂)ₚOR⁶, —(CH₂)ₒ—SO₂-Ph, and —(CH₂)ₒ—O—C₆F₅, where R⁶ and Ph correspond to the definition indicated therefor above, and o and p are identical or different integers between 0 and 10.

Examples of R⁴ as divalent radicals, where is Si-bonded on both sides in accordance with formula (I), are radicals which derive from the monovalent examples stated above for R⁴ by virtue of an additional bond taking place through substitution of a hydrogen atom; examples of such radicals are —(CH₂)—, —CH(CH₃)—, —C(CH₃)₂—, —CH(CH₃)—CH₂—, —C₆H₄—, —CH(Ph)-CH₂—, —C(CF₃)₂—, —(CH₂)—C₆H₄—(CH₂)ₒ—, —(CH₂)ₒ—C₆H₄—C₆H₄—(CH₂)ₒ—, —(CH₂O)ₚ, (CH₂CH₂O)ₒ, —(CH₂)ₒ—Oₓ—C₆H₄—SO₂—C₆H₄—Oₓ—(CH₂)ₒ—, where x is 0 or 1, and Ph, o, and p have the definition stated above.

Preferably, R⁴ comprises a monovalent, SiC-bonded, optionally substituted hydrocarbon radical which has 1 to 18 carbon atoms and is free from aliphatic carbon-carbon multiple bonds, more preferably a monovalent, SiC-bonded hydrocarbon radical which has 1 to 6 carbon atoms and is free from aliphatic carbon-carbon multiple bonds, and more particularly the methyl or phenyl radical.

In certain embodiments, the organopolysiloxane of component (A) preferably contains Si-bonded hydrogen in a range from 0.04 to 1.7 percent by weight (wt %), based on the total weight of the organopolysiloxane.

The molecular weight of the organopolysiloxane of component (A) may likewise vary within wide limits, as for instance between 10² and 10⁶ g/mol. Thus, the organopolysiloxane of component (A) may be, for example, an SiH-functional oligosiloxane of relatively low molecular mass, such as tetramethyldisiloxane, or alternatively may be a silicone resin having SiH groups or a high-polymeric polydimethylsiloxane that possesses SiH groups within the chain or terminally.

The structure of the molecules included in component (A) is also not fixed; in particular, the structure of a SiH-containing organopolysiloxane of relatively high molecular mass, in other words oligomeric or polymeric, may be linear, cyclic, branched, or else resinous, network-like. Linear and cyclic organopolysiloxanes are composed preferably of units of the formula R⁴₃SiO_{1/2}, HR⁴₂SiO_{1/2}, HR⁴SiO_{2/2}, and R⁴₂SiO_{2/2}, with R⁴ having the definition indicated above. Branched and network-like organopolysiloxanes additionally include trifunctional and/or tetrafunctional units, with preference being given to those of the formulae R⁴SiO_{3/2}, HSiO_{3/2}, and SiO_{4/2}, where R⁴ has the definition indicated above.

In some embodiments, component (A) may contain a mixture of molecules including two or more distinct organopolysiloxanes. Particularly preferred is the use of low molecular mass, SiH-functional compounds such as tetrakis(dimethylsiloxy) silane and tetramethylcyclotetrasiloxane, and also of SiH-containing siloxanes of higher molecular mass, such as poly(hydrogenmethyl) siloxane and poly(dimethylhydrogenmethyl) siloxane with a viscosity at 25° C. of 10 to 20 000 mPa·s, or similar SiH-containing compounds in which some of the methyl groups have been replaced by 3,3,3-trifluoropropyl or phenyl groups.

The amount of component (A) in the antimicrobial composition is preferably such that the molar ratio of SiH groups to aliphatically unsaturated groups in the composition is 0.1 to 20, more preferably between 0.3 and 2.0.

In some embodiments, the antimicrobial composition may be of the one-component variety. In other embodiments, the antimicrobial composition may be of the two-component variety. In these embodiments, the antimicrobial composition may be formed by providing a component (B).

Component (B) may comprise an organopolysiloxane compound or another compound. In some embodiments, component (B) may comprise a linear organopolysiloxane compound or another linear compound. In these embodiments, the organopolysiloxane compound may have one or more terminal groups comprising a carbon-carbon multiple bond, which may also be referred to herein as an aliphatic multiple bond. In one such embodiment, the organopolysiloxane compound may comprise an SiC-bonded radical having an aliphatic carbon-carbon multiple bond, which may be referred to herein as an aliphatically unsaturated radical. When component (B) comprises another linear compound, such a compound may comprise aliphatic carbon-carbon multiple bonds.

As noted above, component (B) may comprise an organopolysiloxane compound or another compound. In embodiments where component (B) comprises a silicon-free organic compound, such a compound may comprise at least two aliphatically unsaturated groups. Additionally, it should be noted that component (B) may comprise an organopolysiloxane that has at least two aliphatically unsaturated groups. In certain embodiments, component (B) may comprise a mixture of compounds. In one such embodiment, component (B) may comprise an organopolysiloxane compound that has at least two aliphatically unsaturated groups and a silicon-free organic compound that has at least two aliphatically unsaturated groups. In still other embodiments, component (B) may comprise a mixture of discrete organopolysiloxane compounds and these compounds may each comprise aliphatic carbon-carbon multiple bonds. In these embodiments, the aliphatic carbon-carbon multiple bond may be included in a terminal group or be located in another group of the organopolysiloxane compound.

Examples of silicon-free organic compounds suitable for use in component (B) are 1,3,5-trivinylcyclohexane, 2,3-dimethyl-1,3-butadiene, 7-methyl-3-methylene-1,6-octadiene, 2-methyl-1,3-butadiene, 1,5-hexadiene, 1,7-octadiene, 4,7-methylene-4,7,8,9-tetrahydroindene, methylcyclopentadiene, 5-vinyl-2-norbornene, bicyclo[2.2.1]hepta-2,5-diene, 1,3-diisopropenylbenzene, polybutadiene containing vinyl groups, 1,4-divinylcyclohexane, 1,3,5-triallylbenzene, 1,3,5-trivinylbenzene, 1,2,4-trivinylcyclohexane, 1,3,5-triisopropenylbenzene, 1,4-divinylbenzene, 3-methylhepta-1,5-diene, 3-phenylhexa-1,5-diene, 3-vinylhexa-1,5-diene, and 4,5-dimethyl-4,5-diethylocta-1,7-diene, N,N′-methylenebisacrylamide, 1,1,1-tris(hydroxymethyl) propane triacrylate, 1,1,1-tris(hydroxymethyl) propane trimethacrylate, tripropylene glycol diacrylate, diallyl ether, diallylamine, diallyl carbonate, N, N′-diallylurea, triallylamine, tris(2-methylallyl)amine, 2,4,6-triallyloxy-1,3,5-triazine, triallyl-s-triazine-2,4,6 (1H, 3H,5H)-trione, diallyl malonate, polyethylene glycol diacrylate, polyethylene glycol dimethacrylate, poly(propylene glycol) methacrylate.

Organopolysiloxane compounds known in the art are suitable for use in component (B). Examples of such organopolysiloxanes include, for example, silicone block copolymers having urea segments, silicone block copolymers having amide segments and/or imide segments and/or ester-amide segments and/or polystyrene segments and/or silarylene segments and/or carborane segments, and silicone graft copolymers having ether groups.

Organopolysiloxane compounds suitable for use in component (B) are preferably linear or branched organopolysiloxanes comprising units of the general formula (II)

R⁴ₐR⁵_{b}% SiO_{(4-a-b)/2} (II)
where
   R⁴ independently at each occurrence, is a radical free from aliphatic carbon-carbon multiple bonds,
   R⁵ independently at each occurrence, identically or differently, is a monovalent, substituted or unsubstituted, SiC-bonded hydrocarbon radical having at least one aliphatic carbon-carbon multiple bond,
   a is 0, 1, 2, or 3, and
   b is 0, 1, or 2,
with the proviso that the sum a+b is less than or equal to 3 and there are at least 2 radicals R⁵ per molecule.

R⁴ has the definition indicated above. In some embodiments, R⁵ comprises any desired groups amenable to an addition reaction (hydrosilylation) with an SiH-functional compound.

If R⁵ comprises SiC-bonded, substituted hydrocarbon radicals, preferred substituents are halogen atoms, cyano radicals, and —OR⁶, where R⁶ has the above-stated definition.

Preferably, R⁵ comprises alkenyl and alkynyl groups having 2 to 16 carbon atoms, such as vinyl, allyl, methallyl, 1-propenyl, 5-hexenyl, ethynyl, butadienyl, hexadienyl, cyclopentenyl, cyclopentadienyl, cyclohexenyl, vinylcyclohexylethyl, divinylcyclohexylethyl, norbornenyl, vinylphenyl, and styryl radicals, with vinyl, allyl, and hexenyl radicals being particularly preferred for use.

The molecular weight of the organopolysiloxane compound(s) of component (B) may vary within wide limits, as for instance between 10² and 10⁶ g/mol. Hence, for example, component (B) may comprise an organopolysiloxane that is a relatively low molecular mass, alkenyl-functional oligosiloxane such as, for example, 1,2-divinyltetramethyldisiloxane, or is a polydimethylsiloxane with a molecular weight of 10⁵ g/mol (number average determined by means of NMR) that possesses in-chain or terminal Si-bonded vinyl groups. The structure of the organopolysiloxane of component (B) is also not fixed; in particular, the structure of a siloxane of relatively high molecular mass, in other words an oligomeric or polymeric siloxane, may be linear, cyclic, branched, or else resinous, network-like. Linear and cyclic polysiloxanes are preferably composed of units of the formula R⁴₃SiO_{1/2}, R⁵R⁴₂SiO_{1/2}, R⁵R⁴SiO_{1/2}, and R⁴₂SiO_{2/2}, where R⁴ and R⁵ have the definition indicated above. Branched and network-like polysiloxanes additionally include trifunctional and/or tetrafunctional units, with preference being given to those of the formula R⁴SiO_{3/2}, R⁵SiO_{3/2}, and SiO_{4/2}. Also, as noted above, mixtures of these different organopolysiloxanes may be utilized in component (B).

A preferred organopolysiloxane compound for use in component (B) is a vinyl-functional, substantially linear polydiorganosiloxane having a viscosity of 0.01 to 500 000 Pa·s, more preferably of 0.1 to 100 000 Pa·s, in each case the viscosity being measured at 25° C.

In some embodiments, the antimicrobial composition may contain 30-95 wt %, preferably 30-80 wt %, and more preferably 40-70 wt % of the organopolysiloxane compound(s) of component (B). In other embodiments, the antimicrobial composition may contain 0.1-60 wt %, preferably 0.5-50 wt %, and more preferably 1-30 wt % of the organopolysiloxane(s) of component (A). If the antimicrobial composition comprises an alternative to the organopolysiloxanes of components (A) and (B), then it may be present in 30-95 wt %, preferably 30-80 wt %, more preferably 40-70 wt % in the antimicrobial composition.

In certain embodiments, the antimicrobial composition has a continuous phase and a discontinuous phase. In these embodiments, the antimicrobial composition may be an emulsion. In one embodiment, the continuous phase comprises a polysiloxane network and the discontinuous phase is dispersed in the continuous phase. In some embodiments, the discontinuous phase is uniformly dispersed in the continuous phase. In certain embodiments, the polysiloxane network may be formed by utilizing a single component, such as, for example component (A). In other embodiments, the polysiloxane network is formed by the product of combining an organopolysiloxane from component (A) and organopolysiloxane from component (B). In certain embodiments, the continuous phase may also be formed by utilizing a component (C).

Component (C) may comprise an organopolysiloxane. In some embodiments, the organopolysiloxane may have one or more terminal groups comprising a silicon atom bonded to a hydrogen atom. In other embodiments, the organopolysiloxane may have one or more terminal groups comprising a carbon-carbon multiple bond. In still other embodiments, component (C) may comprise a mixture of organopolysiloxanes. For example, in an embodiment, component C may comprise organopolysiloxane compounds having one or more terminal groups comprising a silicon atom bonded to a hydrogen atom, organopolysiloxane compounds having one or more terminal groups comprising a carbon-carbon multiple bond, and/or organopolysiloxane compounds that do not include any reactive groups. Component (C) may also comprise a reinforcing filler. In other embodiments, component (C) may comprise a nonsilicone oligomeric compound such as, for example, a polyether or a polymeric compounds such as, for example, acrylates, urethanes, polyesters, and copolymers of the same with siloxanes.

Alternatively and instead of utilizing different organopolysiloxanes in components (A) and (B), the polysiloxane network may be formed by providing a single component comprising an organopolysiloxane that simultaneously has aliphatic carbon-carbon multiple bonds and Si-bonded hydrogen atoms.

If an organopolysiloxane that simultaneously has aliphatic carbon-carbon multiple bonds and Si-bonded hydrogen atoms is used, suitable examples are preferably composed of units of the general formula (III), (IV), and (V)

R⁴_{f}SiO_{4/2} (III)

R⁴_{g}R⁵SiO_{3-g/2} (IV)

R⁴ₕHSiO_{3-h/2} (V)
where
   R⁴ and R⁵ have the definitions indicated for them above,
   f is 0, 1, 2, or 3,
   g is 0, 1, or 2, and
   h is 0, 1, or 2,
with the proviso that per molecule there are at least 2 radicals R⁵ and at least 2 Si-bonded hydrogen atoms.

Examples of suitable organopolysiloxanes include those comprising SiO_{4/2}. R⁴₃SiO_{1/2}, R⁴₂R⁵SiO_{1/2}, and R⁴₂HSiO_{1/2} units, known as MQ resins, and these resins may additionally contain R⁴SiO_{3/2} and R⁴₂SiO units, and also linear organopolysiloxanes substantially consisting of R⁴₂R⁵SiO_{1/2}, R⁴₂SiO, and R⁴HSiO units, with R⁴ and R⁵ meeting the aforementioned definition. Organopolysiloxanes that simultaneously have aliphatic carbon-carbon multiple bonds and Si-bonded hydrogen atoms preferably possess an average viscosity of 0.01 to 500 000 Pa·s, more preferably 0.1 to 100 000 Pa·s, in each case at 25° C. Such organopolysiloxanes are preparable by techniques that are known in the art. In addition, Si-bonded hydrogen atoms and aliphatic carbon-carbon multiple bonds can be provided in a single compound.

Preferably, the antimicrobial composition is cross-linkable. In some embodiments, the antimicrobial composition is formed by addition-crosslinking. Crosslinking occurs when the antimicrobial composition is cured. In order to crosslink the organopolysiloxanes provided in the antimicrobial composition, a hydrosilylation catalyst may be provided. The hydrosilyation catalyst may be provided as a portion of one of the components utilized to form the antimicrobial composition.

In the case of a two-component silicone compositions, the two components may comprise all constituents referred to above in any desired combinations, generally with the proviso that one component does not simultaneously comprise organopolysiloxane compounds with aliphatic multiple bonds, organopolysiloxane compounds with Si-bonded hydrogen atoms, and the hydrosilylation catalyst. Thus, in the case of a two-component composition, the hydrosilyation catalyst may be provided as a portion of component (A) or component (B).

Hydrosilylation catalysts known in the art are suitable for use in the antimicrobial composition. The hydrosilylation catalyst may include a platinum-group metal such as, for example, platinum, rhodium, ruthenium, palladium, osmium, or iridium, or may be an organometallic compound, or a combination thereof. Suitable examples of hydrosilylation catalysts are compounds such as hexachloroplatinic (IV) acid, platinum dichloride, platinum acetylacetonate, and complexes of said compounds encapsulated in a matrix or in a core/shell-like structure. Other suitable platinum complexes with a low molecular weight of organopolysiloxanes include 1,3-diethenyl-1,1,3,3-tetramethyldisiloxane complexes with platinum. Other examples of suitable hydrosilation catalysts are platinum-phosphite complexes, platinum-phosphine complexes, or alkylplatinum complexes such as derivatives of cyclopentadienyltrimethylplatinum (IV), cyclooctadienyldimethylplatinum (II), or diketonato complexes, such as bisacetylacetonatoplatinum (II), for example. In certain embodiments, the platinum-containing compound may be encapsulated within a resin matrix.

The concentration of catalyst for catalyzing the hydrosilylation crosslinking reaction may be in an amount between 0.1 and 1000 parts per million (ppm), 0.5 and 100 ppm, or 1 and 25 ppm of the platinum group metal, depending on the total weight of the antimicrobial composition.

In the embodiments described above, the antimicrobial composition may be provided as a gel. In these embodiments, the gel has a crosslinked structure. A crosslinked structure can form when the total number of reacting groups is greater than 4. Thus, for a gel formed by a platinum catalyzed hydrosilylation reaction, crosslinking can happen, for example, between a first organopolysiloxane containing more than two Si-bonded hydrogen atoms and a second organopolysiloxane with at least two reactive aliphatically unsaturated groups or alternatively between a first organopolysiloxane containing two Si-bonded hydrogen atoms and a second organopolysiloxane with more than two aliphatically unsaturated radicals. Preferably, in these embodiments, the first organopolysiloxane and the second organopolysiloxane are crosslinked to the gel point of the mixture. In such embodiments and before curing, the antimicrobial composition may exhibit a viscosity of 50-100,000 centipoise.

The antimicrobial composition comprises a silver-containing antimicrobial agent. In certain embodiments, the silver-containing antimicrobial agent is a silver salt with antimicrobial properties. The silver-containing antimicrobial agent can be selected from the group comprising Ag₂SO₄, Ag₂SO₃, AgNO₃, Ag₂CO₃, Ag₃PO₄, silver zirconium, and/or organic silver salts, such as silver citrate, silver acetate, silver lactate and/or combinations or mixtures thereof. Other compounds suitable for providing silver ions when desired are also suitable as the silver-containing antimicrobial agent. The silver-containing antimicrobial agent may be provided in the antimicrobial composition at about 1 weight percent (wt %) to about 30 wt %, preferably, about 2 to 20 wt %, in all cases based on the total weight of the composition. The silver-containing antimicrobial agent may be provided as a portion of component (A), component (B), component (C), in two or more of these components, or as a separate addition to the composition.

The antimicrobial composition comprises an excipient. The antimicrobial composition may comprise 0.5 wt % or more of the excipient, based on the total weight of the composition. Preferably, the antimicrobial composition comprises 0.5 to 15 wt % of the excipient, based on the total weight of the composition. More preferably, the antimicrobial composition comprises 0.5 to 10 wt % of the excipient, based on the total weight of the composition. The excipient may be provided as a portion of component (A), component (B), component (C), or in two or more of these components.

Preferably, the weight percent of the excipient in the antimicrobial composition is greater than the weight percent of the silver-containing antimicrobial agent in the antimicrobial composition, based on the total weight of the composition. For example, the weight percent of the excipient present in the antimicrobial composition may be 2 or more times greater than the weight percent of the silver-containing antimicrobial agent. In an embodiment, the weight percent of the excipient present in the antimicrobial composition is 2 to 10 times greater than the weight percent of the silver-containing antimicrobial agent.

In some embodiments, the excipient is an alcohol and comprises one or more hydroxyl groups. In other embodiments, the excipient may be a glycol. In these embodiments, the excipient has two or more hydroxyl groups and two hydroxyl groups of the two or more hydroxyl groups may be connected to different carbon atoms. In one such embodiment, the two hydroxyl groups of the two or more hydroxyl groups are end groups. In some embodiments, the excipient is a polyether.

In an embodiment, the excipient has a number average molecular weight of 100 g/mol or more. In other embodiments, the excipient may have a number average molecular weight of 300 g/mol or more. Preferably, the excipient has a relatively low number average molecular weight. In one such embodiment, the excipient has a number average molecular weight of 300-2,000 g/mol. In another embodiment, the number average molecular weight of the excipient is 300-1000 g/mol. Preferably, in these embodiments, the number average molecular weight of the excipient is 300-600 g/mol.

In some embodiments, the excipient may be selected from the group consisting of poly(propylene glycol) having a number average molecular weight of 400-2000 g/mol, 1,2-propanediol, di(propylene glycol), di(propylene glycol) monomethyl ether, di(propylene glycol) dimethyl ether, organopolysiloxane polyoxyalkylenes, copolymers, and mixtures thereof. In certain embodiments, it may be preferred that the excipient is poly(propylene glycol) having a number average molecular weight of 400-2000 g/mol. Preferably, in these embodiments, the excipient is poly(propylene glycol) having a number average molecular weight of 400 g/mol. In other embodiments, it may be preferred that the excipient is an organopolysiloxane polyoxyalkylene. Preferably, in these embodiments, the excipient is an organopolysiloxane polyoxyalkylene of the general formula:

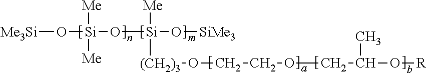

In certain embodiments, the organopolysiloxane polyoxyalkylene may be covalently bonded. An example of a commercially available organopolysiloxane polyoxyalkylene suitable as the excipient is sold under the name Belsil® OW 1500 and by Wacker Chemie AG.

As noted above, the antimicrobial composition has a discontinuous phase. The antimicrobial agent and the excipient are present in the discontinuous phase. While not wishing to be bound by a particular theory, it is believed that the silver-containing compounds in traditional antimicrobial compositions disassociate and form an oxide of silver or elemental silver in the presence of air. The resulting disassociation creates silver ions, which can be reduced and/or oxidized, leading to an undesirable discoloration of the composition. Typically, the discoloration is of a dark color such as black or brown. In the present antimicrobial composition, the excipient inhibits the silver-containing antimicrobial agent from forming elemental silver or silver oxide in the presence of air. Preferably, the excipient inhibits the silver-containing antimicrobial agent from forming elemental silver or silver oxide to the extent that the antimicrobial composition does not exhibit an observable change in color after exposure to air having a relative humidity of 50% at 25° C. for 24 hours. In certain embodiments, the antimicrobial composition does not exhibit an observable change in color after exposure to air having a relative humidity of 50% at 25° C. for 168 hours. Moreover, when the antimicrobial composition is provided as a gel, the composition may not exhibit an observable change in color after exposure to air having a relative humidity of 50% at 25° C. for 12 months. Furthermore, in embodiments where the antimicrobial composition does exhibit an observable change in color, the change in color is significantly reduced over the color change observed in the known compositions. Additionally, any change in color results in a color that is exhibited by the antimicrobial composition that is visually pleasing.

The antimicrobial composition may comprise one or more additives, which may be provided as a portion of component (A), component (B), or component (C). For example, as mentioned above, the anti-microbial composition may comprise a reinforcing filler. Suitable reinforcing fillers include fumed or precipitated silicas having BET surface areas of at least 50 m²/g, carbon blacks, activated carbons such as furnace black and acetylene black, or mixtures thereof. The stated silica fillers may have a hydrophilic character or may have been made hydrophobic by known methods. The amount of reinforcing filler in the antimicrobial composition may be within the range from 0 to 70 wt %, preferably 0 to 50 wt %, based on the total weight of the antimicrobial composition.

In certain embodiments, it is preferred that the filler utilized is surface-treated. The surface treatment is obtained by the methods known in the art for hydrophobizing finely divided fillers. As a result of a surface treatment, the filler utilized may have a carbon content of at least 0.01 up to a maximum of 20 wt %, preferably between 0.1 and 10 wt %, more preferably between 0.5 to 5 wt %. Preferably, in these embodiments, the filler is a surface-treated silica having 0.01 to 2 wt % of Si-bonded, aliphatically unsaturated groups. These groups are, for example, Si-bonded vinyl groups. In the antimicrobial composition, the filler is provided as a single species or as a mixture of two or more finely divided filler(s).

Further additives may be provided in the antimicrobial composition in a fraction of up to 70 wt %, preferably 0.0001 to 40 wt %, based on the total weight of the composition. These additives may be, for example, inert fillers, resinous polyorganosiloxanes, different from the siloxanes described above, reinforcing and nonreinforcing fillers, fungicides, fragrances, rheological additives, corrosion inhibitors, oxidation inhibitors, light stabilizers, flame retardants, and agents for influencing the electrical properties, dispersing assistants, solvents, adhesion promoters, pigments, dyes, plasticizers, organic polymers, heat stabilizers, etc. These include additives, such as finely ground quartz, diatomaceous earth, clays, chalk, lithopone, carbon blacks, graphite, metal oxides, metal carbonates, metal sulfates, metal salts of carboxylic acids, metal dusts, fibers, such as glass fibers, polymeric fibers, polymeric powders, metal dusts, dyes, pigments, etc. Additional fillers may be heat-conducting or electrically conducting. A combination of fillers with different particle sizes and different particle size distributions may also be utilized.

Further, the antimicrobial composition may comprise additional additives. For example, the antimicrobial composition may comprise one or more solvents and/or one or more inhibitors. In other embodiments, the antimicrobial composition may comprise a hydrophilic additive compound, which may enhance silver release from the composition. Hydrophilic additive compounds known in the art are suitable for use in the antimicrobial composition. These hydrophilic additive compounds are known to make antimicrobial compositions, when crosslinked, swell by at least 5% after 24 hours in a water solution containing 8.298 g/L of sodium chloride and 0.368 g/L of calcium chloride dihydrate, as measured by the free swell absoption method.

When provided as a gel, the antimicrobial composition may also function as an adhesive. In these embodiments, the antimicrobial composition may be utilized as the adhesive portion of a wound care dressing. In this embodiment, the tack exhibited by the gel may be greater than 50 grams of force (gf). Preferably, the tack exhibited by the gel is greater than 100 gf. However, the tack must not be so strong that the skin of the user is damaged when the dressing is removed. Thus, in some embodiments, the tack exhibited by the gel is less than 800 gf. In these embodiments, the tack exhibited by the gel may be 50-800 gf. The tack exhibited by the gel can be measured by known methods. For example, the tack of the gel can be measured with a TA.XT Plus Texture Analyzer using a TA-57R probe and a TA-303 apparatus.

In addition to a suitable tack, it is preferred that the gel is cohesive. A cohesive gel does not break apart when removed from a surface it has been adhered to. In some embodiments, the gel may exhibit a post-cure penetration hardness of 25-500 1/10 mm measured according to DIN ISO 2137 using a hollow cone of 62.5 grams for 60 seconds after curing for 60 minutes at 120° C.

The gel can be combined with or incorporated into a wound care dressing prior to the application of the dressing to a user or applied directly on the user. In such applications, in certain embodiments, the gel does not exhibit an observable change in color for extended periods of time. For example, the gel may not exhibit an observable change in color after exposure to air having a relative humidity of 50% at 25° C. for 24 hours. In fact, in gel form, the antimicrobial composition may not exhibit an observable change in color after exposure to air having a relative humidity of 50% at 25° C. for 365 days. However, in other embodiments, where the antimicrobial composition is in a gel form, the composition may exhibit an observable color change. In these embodiments, the change in color is significantly reduced over the color change observed in the known compositions. Furthermore, any change in color results in a color that is exhibited by the antimicrobial composition that is visually pleasing.

The antimicrobial composition may be formed as a gel after being applied to a substrate. In some embodiments, the antimicrobial composition may coat a substrate such as, for example, a dressing. In these embodiments, the antimicrobial composition may be cast and cured on the substrate to form the gel. The antimicrobial composition can be applied to the substrate to provide any desired thickness, pattern, or morphology. Suitable substrates are known in the art.

In advance of forming the gel, the antimicrobial composition may be made by preparing component (A). Preferably, component (A) comprises the organopolysiloxane(s) described above for component (A). Additionally, component (A) may comprise a silver-containing antimicrobial agent, an excipient, a hydrosilyation catalyst, and/or one or more additives. When included in component (A), the silver-containing antimicrobial agent, excipient, hydrosilyation catalyst, and the one or more additives may be mixed with the organopolysiloxane(s) to form a mixture. Mixing can be done at a predetermined rate, for a predetemined period of time, and utilizing commercially available mixing devices such as, for example, a Speedmixer® or a Dispermat® fitted with a dissolver blade. The silver-containing antimicrobial agent, excipient, hydrosilyation catalyst, and the one or more additives may be as described above.

In certain embodiments, the antimicrobial composition may be made by preparing component (B). Preferably, component (B) comprises the organopolysiloxane described above for component (B). Additionally, component (B) may comprise the silver-containing antimicrobial agent, excipient, hydrosilyation catalyst, and/or one or more additives. When included in component (B), the silver-containing antimicrobial agent, excipient, hydrosilyation catalyst, and the one or more additives may be mixed with the organopolysiloxane to form a mixture. Mixing can be done at a predetermined rate, for a predetemined period of time, and utilizing commercially available mixing devices such as, for example, a the mixing devices mentioned above. The silver-containing antimicrobial agent, excipient, hydrosilyation catalyst, and the one or more additives may be as described above.

In embodiments where the antimicrobial composition comprises component (C), the antimicrobial composition is made by preparing component (C). In these embodiments, the antimicrobial composition may be made by preparing three mixtures and combining those mixtures. Preferably, component (C) comprises an organopolysiloxane like ones described above for component (C). Additionally, component (C) may comprise the silver-containing antimicrobial agent, excipient, hydrosilyation catalyst, and/or one or more additives. When included in component (C), the silver-containing antimicrobial agent, excipient, hydrosilyation catalyst, and the one or more additives may be mixed with the organopolysiloxane to form a mixture. Thus, in these embodiments, the antimicrobial composition may be made by initially preparing three mixtures. Mixing can be done at a predetermined rate, for a predetermined period of time, and utilizing commercially available mixing devices as described above. The silver-containing antimicrobial agent, excipient, hydrosilylation catalyst, and the one or more additives may be as described above.

In certain embodiments, and prior to coating the substrate, component (A) and component (B) may be mixed to form a mixture. Mixing can be done at a predetermined rate, for a predetermined period of time, and utilizing commercially available mixing devices such as, for example, the mixing devices mentioned above. In some embodiments, the mixture may also include component (C). If not included in components (A), (B) or (C) or if additional amounts are desired to be included in the antimicrobial composition, the silver-containing antimicrobial agent, excipient, hydrosilyation catalyst, and/or one or more additives can be added to the mixture. The addition of the silver-containing antimicrobial agent, excipient, hydrosilyation catalyst, and/or one or more additives to the mixture can be achieved at the time of mixing component (A), component (B), component (C) or can occur simultaneously or sequentially by way of further mixing. After mixing, one or more portions of the substrate can be coated with the antimicrobial composition and the composition can be cured at a predetermined temperature and for a predetermined period of time. For example, the mixture can be cured at a temperature of 40-140° C., preferably 60-130° C., for 5 seconds to 2 hours, preferably 10 seconds to 30 minutes.

In certain embodiments, component (A) is prepared such that it comprises an organopolysiloxane having one or more groups comprising a silicon atom bonded to a hydrogen atom. In this embodiment, the antimicrobial composition is made by mixing a silver-containing antimicrobial agent with the organopolysiloxane having one or more groups comprising a silicon atom bonded to a hydrogen atom and an excipient. Preferably, the excipient, comprises a polyether having an average molecular weight of 300-2,000 g/mol or an organopolysiloxane polyoxyalkylene. In an embodiment, component (B) is prepared such that it comprises an organopolysiloxane having one or more groups organopolysiloxane having one or more terminal groups comprising a carbon-carbon multiple bond. Preferably, component (B) also comprises one or more additives such as, for example, a reinforcing filler. In this embodiment, a mixture is formed. The mixture comprises component (A), component (B), the silver-containing antimicrobial agent, the excipient, and a hydrosilyation catalyst. An antimicrobial gel is formed by curing the mixture.

### EXAMPLES

The following examples are presented solely for the purpose of further illustrating and disclosing the embodiments of the antimicrobial composition. Examples of the antimicrobial composition include Examples 1-16 and are described below. Comparative Examples 1 and 2, which are not part of the invention, are also described below.

### Comparative Example 1

A composition was formed by mixing 3 grams of a component (A) comprising an organopolysiloxane having a molecular weight of about 1000 g/mol and one or more groups comprising a silicon atom bonded to a hydrogen atom with a 0.5 grams of a mixture of silver sulfate and pyrogenic silica for 5 minutes using a SpeedMixer® at 2350 rpm. The mixture of silver sulfate and pyrogenic silica was 99% silver sulfate and 1% pyrogenic silica, based on the total weight of the mixture. The composition was initally white in color but was observed to change color rapidly when exposed to air at temperature of approximately 25° C. After 60 seconds of being exposed to air at 25° C., the composition was a dark brown to black color.

### Example 1

An antimicrobial composition was formed by mixing 3 grams of a component (A) comprising an organopolysiloxane having a molecular weight of about 1000 g/mol and one or more groups comprising a silicon atom bonded to a hydrogen atom was mixed with poly(propylene glycol) having a number average molecular weight of approximately 400 g/mol and a 0.5 grams of a mixture of silver sulfate and pyrogenic silica for 5 min minutes using a SpeedMixer® at 2350 rpm. The mixture of silver sulfate and pyrogenic silica was as described above in Comparative Example 1. The resulting antimicrobial composition included a continuous phase and a discontinuous phase. The continuous phase comprised the organopolysiloxane and the discontinuous phase comprised the silver sulfate and the poly(propylene glycol). The presence of the silver sulfate and the poly(propylene glycol) in the discontinuous phase was confirmed by optical microscopy and was evident by the light scattering and white color of the composition. The color of the antimicrobial composition did not undergo an observable change after being exposed to air at temperature of 25° C. for 60 seconds.

### Comparative Example 2

A mixture of silver sulfate and pyrogenic silica was mixed with a component (B) comprising an organopolysiloxane having one or more terminal groups comprising a carbon-carbon multiple bond for 5 minutes using a SpeedMixer® at 2350 rpm. The mixture of silver sulfate and pyrogenic silica was as described above in Comparative Example 1. A component (A) comprising an organopolysiloxane having one or more groups comprising a silicon atom bonded to a hydrogen atom was then added thereto using a SpeedMixer® at 2350 rpm for 30 seconds. The resulting composition comprised 2 wt % of the silver sulfate and pyrogenic silica mixture with the remaining portion of the composition being formed from equal parts of component (A) and component (B). Before curing, the composition was white. A portion of the composition was cast on a biaxially-oriented PET substrate to achieve a wet film thickness of 15 mils and then cured at 120° C. for 12 minutes. The remaining portion of the composition was allowed to cure by exposure to air at a temperature of approximately 25° C. for 24 hours. After curing, the composition was a gel that was appeared brown in color.

### Example 2

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel that exhibited no observable change in color.

### Example 3

Poly(propylene glycol) having a number average molecular weight of approximately 725 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly yellow tint.

### Example 4

Poly(propylene glycol) having a number average molecular weight of approximately 1000 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly yellow tint.

### Example 5

Poly(propylene glycol) having a number average molecular weight of approximately 2000 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly yellow tint.

### Example 6

Poly(ethylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % poly(ethylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly pink tint.

### Example 7

1,2-propanediol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % 1,2-propanediol with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly yellow tint.

### Example 8

Di(propylene glycol), a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % di(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel that was observed to be colorless.

### Example 9

Di(propylene glycol) monomethyl ether, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % di(propylene glycol) monomethyl ether with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 25° C. for 24 hours. After curing, the antimicrobial composition was a gel exhibiting a yellowish tint.

### Example 10

Di(propylene glycol) dimethyl ether, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 5 wt % di(propylene glycol) dimethyl ether with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a yellowish tint that was more intense than the tint observed in Example 7.

### Example 11

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 2.5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel that exhibited no observable change in color.

### Example 12

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 1.0 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel that exhibited no observable change in color.

### Example 13

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 2 wt % of the silver sulfate and pyrogenic silica mixture and 0.5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly pink tint.

### Example 14

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 5 wt % of the silver sulfate and pyrogenic silica mixture and 0.5 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly pink tint.

### Example 15

Poly(propylene glycol) having a number average molecular weight of approximately 400 g/mol, a mixture of silver sulfate and pyrogenic silica, and a component (B) comprising an organopolysiloxane having one or more terminal groups comprising a carbon-carbon multiple bond were mixed for 5 minutes using a SpeedMixer® at 2350 rpm to form a first mixture. The mixture of silver sulfate and pyrogenic silica was as described above in Comparative Example 1. A component (A) comprising an organopolysiloxane having one or more groups comprising a silicon atom bonded to a hydrogen atom was then added to the first mixture using a SpeedMixer® at 2350 rpm for 30 seconds. The resulting antimicrobial composition comprised 10 wt % of the silver sulfate and pyrogenic silica mixture and 1.0 wt % poly(propylene glycol) with the remaining portion of the antimicrobial composition being formed from equal parts of component (A) and component (B). Before curing, the antimicrobial composition was white. A portion of the antimicrobial composition was cast on a biaxially-oriented PET substrate to achieve a wet film thickness of 15 mils and then cured at 120° C. for 12 minutes. The remaining portion of the composition was allowed to cure by exposure to air at a temperature of approximately 20° C. for 24 hours. After curing, the antimicrobial composition was a gel exhibiting a slightly pink tint.

### Example 16

A first mixture was formed. The first mixture comprised an organopolysiloxane polyoxyalkylene sold as Belsil® OW 1500 by Wacker Chemie AG and a component (B) comprising an organopolysiloxane having one or more terminal groups comprising a carbon-carbon multiple bond. The first mixture was formed using a SpeedMixer® at 2350 rpm. A second mixture of silver sulfate, pyrogenic silica, and the component (B) was formed using a SpeedMixer® at 2350 rpm. An antimicrobial composition was formed, using a SpeedMixer® at 2350 rpm for 30 seconds, by mixing the first mixture, second mixture, and a component (A) comprising an organopolysiloxane having one or more groups comprising a silicon atom bonded to a hydrogen atom. The resulting antimicrobial composition comprised 3.1 parts of the silver sulfate, 0.6 parts pyrogenic silica, 14.3 parts Belsil® OW 1500 with the remaining portion of the antimicrobial composition being formed from equal parts of component (A) and component (B). Before curing, the antimicrobial composition was white. Portions of the antimicrobial composition were cast, drawn down, and then cured at 120° C. for 12 minutes. After curing, each portion of the antimicrobial composition was a gel having a thickness of between 8 and 9 mils. The gel portions did not exhibit an observable color change.

From the foregoing detailed description, it will be apparent that various modifications, additions, and other alternative embodiments are possible without departing from the true scope and spirit. The embodiments and examples discussed herein were chosen and described to provide the best illustration of the principles of the invention and its practical application to thereby enable one of ordinary skill in the art to use the invention in various embodiments and with various modifications as are suited to the particular use contemplated. As should be appreciated, all such modifications and variations are within the scope of the invention.

1. An antimicrobial composition, comprising:
a component (A) including an organopolysiloxane having one or more groups comprising a silicon atom bonded to a hydrogen atom;
a silver-containing antimicrobial agent; and
an excipient,
wherein the antimicrobial composition has a discontinuous phase and the antimicrobial agent and the excipient are present in the discontinuous phase.

2. The composition of claim 1, wherein the silver-containing antimicrobial agent is silver salt.

3. The composition of claim 1, wherein the excipient is an alcohol, a glycol, a polyether, or an organopolysiloxane polyoxyalkylene.

4. The composition of claim 1, wherein the excipient has an average molecular weight of 300 g/mol or more.

5. The composition of claim 1, further comprising a hydrophilic additive compound.

6. The composition of claim 1, wherein the excipient inhibits the silver-containing antimicrobial agent from forming elemental silver or silver oxide in the presence of air having a relative humidity of 50% at 20° C.

7. The composition of claim 1, wherein the composition does not exhibit an observable change in color after exposure to air having a relative humidity of 50% at 20° C. for 24 hours.

8. The composition of claim 1, further comprising a component (B) that includes an organopolysiloxane compound having one or more terminal groups comprising a carbon-carbon multiple bond.

9. The composition of claim 1, wherein the weight percent of the excipient is greater than the weight percent of the silver-containing antimicrobial agent, based on the total weight of the composition.

10. The composition of claim 1, wherein the composition comprises 0.5 wt % or more of the excipient, based on the total weight of the composition.

11. The composition of claim 3, wherein the excipient is a polyether having an average molecular weight of 300-2,000 g/mol.

12. The composition of claim 4, wherein the average molecular weight of the excipient is 300-2,000 g/mol.

13. The composition of claim 8, wherein the antimicrobial composition has a continuous phase comprising an polysiloxane network formed by the product of combining component (A) and component (B).

14. The composition of claim 8, further comprising a hydrosilyation catalyst provided as a portion of component (A) or component (B).

15. The composition of claim 8, further comprising a component (C), wherein component (C) comprises an organopolysiloxane and a filler.

16. The composition of claim 9, wherein the weight percent of the excipient in the composition is 2 or more times greater than that of the silver-containing antimicrobial agent.

17. The composition of claim 10, wherein the composition comprises from 0.5 to 15 wt % of the excipient, based on the total weight of the composition.

18. The composition of claim 11, wherein the average molecular weight of the excipient is 300-1000 g/mol.

19. The composition of claim 16, wherein weight percent of the excipient is 2 to 10 times greater than that of the silver-containing antimicrobial agent.

20. A gel adhesive comprising the composition of claim 1.
